(19) United States
(12) Patent Application Publication
(10) Pub. No.: US 2004/0241879 A1
(43) Pub. Date: Dec. 2, 2004

(54) Assay device and method

(76) Inventor: Joseph R. Robinson, New York, NY (US)

Correspondence Address: DARBY & DARBY P.C., P. O. BOX 5257, NEW YORK, NY, 10150-5257, US

(21) Appl. No.: 10/859,630

(22) Filed: Jun. 2, 2004

Related U.S. Application Data
(60) Provisional application No. 60/475,278, filed on Jun. 2, 2003.

(51) Int. Cl. G01N 33/558

(52) U.S. Cl. 436/514

(57) ABSTRACT

The present invention provides a test device for detecting the presence or absence of a selected analyte in a liquid sample. The test device includes a reagent member having a body, a first color-labeled binding reagent specific for a first binding site of said analyte; wherein said body is adapted to retain said first color-labeled specific binding reagent when said body and said first color-labeled binding reagent are dry, and to release said first color-labeled specific binding reagent when said body and said first color-labeled specific binding reagents are moist, a porous carrier; and a detection zone having a second color-labeled binding reagent specific for a second binding site of said analyte, wherein said second color-labeled specific binding reagent is immobilized in said detection zone, wherein said first specific binding site and said second binding site are different, and wherein in said first and second labeled specific binding reagents are capable of forming a third color-labeled complex with said analyte, said complex comprising said first color-labeled specific binding reagent, said analyte, and said second color-labeled specific binding reagent; wherein said first color, said second color-label, and said third color are visually distinguishable from one another by the human eye; wherein said reagent member, porous carrier, and detection zone are arranged so that a fluid applied to said test device would travel sequentially from said reagent member to said porous carrier and to said detection zone. The test device is typically dry before use and moist during use.

### FIELD OF THE INVENTION

The present invention relates to assays involving specific binding, especially immunoassays. The test devices and methods of the present invention are suitable for quantitative assays as well as for qualitative assays. The invention particularly relates to analytical devices which are suitable for use in the home, clinic or doctor's office, which are intended to give an analytical result rapidly, and which require the minimum degree of skill and involvement from the user. The use of test devices in the home to test for pregnancy and fertile period (ovulation) is now commonplace.

### BACKGROUND OF THE INVENTION

Many previous reagent-impregnated test strips used in specific binding assays, such as immunoassays, have required the use of a single labeled specific binding reagent for the analyte and a single unlabeled specific binding reagent for the analyte. The former is located upstream from a detection zone and is mobile, while the latter is located in the detection zone and is permanently immobilized. A sample suspected of containing an analyte is applied to the device. Any analyte in the sample binds with the labeled mobile specific binding reagent and becomes labeled. These labeled complexes travel with the sample downstream to the detection zone where they complex further with the unlabeled, permanently immobilized specific binding reagent. The presence of these complexes in the detection zone indicates the presence of the analyte in the sample. See, e.g., U.S. Pat. Nos. 6,228,660 and 6,352,862.

Other earlier sandwich immunoassay devices utilize a colloidal gold labeled antiligand reagent and antiligand bound solid phase capture particles which are combined with the sample. The reactants are incubated and placed onto a porous film wherein the pore size of the porous film is such that the solid phase particles are retained on the surface of the film but large enough that the unbound colloidal gold reagents can pass through. The particles on the membrane are visually inspected for color to determine the presence or absence of the analyte. See U.S. Pat. No. 4,853,335.

Another earlier immunoassay device utilizes marking elements and particles which do not affect evaluation. Each of these include bispecific antibodies for the suspected analyte. The device includes a porous catching section that has a pore diameter that is smaller than the diameter of the particles. When the analyte is present, a biochemical reaction takes place between the marking elements and the particles producing a reaction product. The pore size of the catching section is smaller than the size of the particles and smaller than the size of the reaction product. The particles and the reaction product are caught by the catching section, but the marking element can pass through the catching section. Therefore, a visual inspection of the catching section will indicate the presence or the absence of the analyte in the sample. See European Patent Application No. EP 0962771.

### BRIEF DESCRIPTION OF THE DRAWINGS

FIG. 1 is an isometric view of a test device according to the present invention.

FIG. 2 is a cross-sectional side elevation of a test device according to the present invention.

FIG. 3 is a side view of a test device according to the present invention.

### SUMMARY OF THE INVENTION

The present invention provides a test device for detecting the presence or absence of a selected analyte in a liquid sample. The test device includes a reagent member, a porous carrier, and a detection zone. The reagent member includes a body and a first color-labeled binding reagent specific for a first binding site of the analyte The reagent body is adapted to retain the first color-labeled specific binding reagent when the body and the first color-labeled binding reagent are dry, and to release the first color-labeled specific binding reagent when the body and the binding reagent are moist. The detection zone includes a second color-labeled binding reagent specific for a second binding site of the analyte. Preferably, the second color-labeled binding reagent is immobilized, and more preferably, is permanently immobilized in the detection zone. The first and second labeled specific binding reagents are capable of forming a third color-labeled complex with the analyte with the complex including the first color-labeled specific binding reagent, the analyte, and the second color-labeled specific binding reagent. The first specific binding site and second binding sites are different. The detection zone may be a section of the porous carrier or may be separate from and in fluid communication with the porous carrier. The reagent member, porous carrier, and detection zone are arranged so that a fluid applied to the test device would travel sequentially from the reagent member to the porous carrier and to the detection zone. The test device is typically dry before use and moist during use. The first and second color-labels can interact to give the three component complex a third, different color. Alternatively, the first color may mask the second color so that a positive test is indicated by the presence of the first color and the absence or diminishment of the second color.

The test device may further include a sample receiving member arranged so that a fluid applied to the test device would travel sequentially from the sample receiving member to the reagent member.

The test device may also include a control zone arranged so that a fluid applied to the test device would travel sequentially from the reagent member to the porous carrier, to the detection zone, and to the control zone. The control zone may include a porous barrier having an average pore size smaller than the diameter of the first color-labeled specific binding reagent. Another type of control zone could include an immobilized binding reagent capable of binding to the first color-labeled specific binding reagent. The control zone may be a section of said porous carrier or may be separate from and in fluid communication with the porous carrier.

The present invention also provides a test device as described above for detecting the presence or absence of human chorionic gonadotrophin (hCG) in urine. Specific components of one preferred embodiment of a test device for detecting hCG include a first colored latex particle labeled anti-hCG monoclonal antibody for a first antibody binding site of hCG and a second colored latex particle labeled different anti-hCG antibody specific for a second antibody binding site of hCG, which together with hCG form a third colored complex. The porous carrier may be a nitrocellulose porous carrier. The second color-labeled anti-hCG antibody may be immobilized, and preferably, permanently immobilized, in this zone. The colors of the particles may be different colors or may be distinguishable shades of the same color but differences in color should be visually distinguishable from one another by the eye of a normal human being. In one embodiment, the first, second, and third colors are different. The third color is formed by the interaction of the first and second colors. In an alternate embodiment, the first and second colors are different, but third color is the same as the first color. In this latter embodiment, the third color, which is the same as the first color, may appear because the first color masks the second color in the complexes.

The present invention also provides a method of detecting the presence or absence of an analyte in a liquid sample by applying the liquid sample to the test devices described herein, whereby third color complexes form and are retained at the detection zone, and at least a portion of the uncomplexed first color-labeled specific binding reagents pass through the detection zone; and (b) detecting the presence or absence of the third color complexes at the detection zone. The presence of the complexes in the detection zone indicates the presence of the said analyte in the sample.

### DETAILED DESCRIPTION OF THE INVENTION

The assay devices and methods of the present invention may be to detect a wide variety of analytes by choosing appropriate specific binding reagents. The analytes can be, for example, proteins, haptens, immunoglobulins, hormones, polynucleotides, steroids, drugs, or infectious disease agents (e.g. of biological, chemical, or biochemical origin). Examples of analytes include, but are not limited to, hCG (human chorionic gonadotrophin) (a hormone whose presence indicates pregnancy), LH (lutinizing hormone) (a hormone whose presence indicates ovulation), infectious disease agents, E-3-G (estrone-3-glucoronide) and P-3-G (pregnanediol-3-glucoronide). The former three are suitably analyzed with the present device in a sandwich assay, while the latter two are suitably analyzed in a competition assay.

The present devices and assays can also be adapted, based upon the detailed description herein, to detect simultaneously or sequentially more than one analyte in a sample, which can also have significant clinical utility. For example, the ratio of the levels of apolipoproteins A₁ and B can indicate susceptibility to coronary heart disease, while the ratio of the levels of glycated hemoglobin (HbA) to unglycated (HbAo) or total (Hb) hemoglobin can aid in the management of diabetes. Test devices may also be configured, based upon the detailed disclosure herein, to measure two steroids simultaneously, such as, for example, E-3-G and P-3-G. A test could also be configured, based upon the detailed disclosure herein, to detect the presence of various different sereotypes of one bacterium, or to detect the presence of soluble serological markers in humans. For example, a multiple analyte test for the detection of different serotypes of Streptococcus can be prepared for groups A, B, C and D. A cocktail of monoclonal antibodies labeled with labels of different colors, each of which would form a different third color specific for various pathologically important group serotypes, or a polyclonal antiserum raised against a particular Streptococcal group, could be placed onto a reagent member and one or more detection zones having the same or different second color labels and the appropriate binding reagents, as described herein, to detect each could be placed on the device.

The reagent member may be one individual reagent sub-member or more than one reagent sub-members each containing one or more specific binding reagents. The reagent member may be composed of, for example, a fibrous material or pad-like structure that may be woven or non-woven, such as, for example, a fiberglass pad, with openings or interstitial spaces which act as pores, and may be termed pores herein. Alternatively, the reagent member may be a natural or synthetic porous material such as a macroporous body. The reagent member may be a section of the porous carrier or may be a separate component. The use of a separate component reagent member may facilitate the ease with which the labeled components are taken up by the liquid sample as compared to if the labeled components are incorporated as pre-dosed reagents on the porous carrier. Manufacturing is also facilitated by having the reagent member as a separate component that is placed next to and slightly overlapping the porous carrier during manufacture of the test device.

Preferably, the reagent member is in direct moisture-conductive contact with the porous carrier, and the detection zone, whether on the porous carrier or a separate component. Alternatively, the reagent member may be spaced away from the region of contact between the porous carrier and the reagent member. Preferably in the latter embodiment, the quantity of liquid sample required to saturate the reagent member is not less than the quantity of liquid sample capable of being absorbed by the mass of porous carrier linking the reagent member and the detection zone. Preferably in either instance, the liquid capacity of the reagent member is at least equal to the liquid capacity of the working portion of the porous carrier.

Preferably, the reagent member has openings, spaces, or pores (all included in the term Pores herein) that are at least 10 times greater than the maximum size of the largest first color-label used in the device. Larger pore sizes or openings give better release of the labeled reagent.

The reagent member preferably is not protein-binding or is easily blockable by means of reagents such as BSA or PVA, to minimize non-specific binding and to facilitate free movement of the labeled reagents after the reagent member has become moistened with the liquid sample.

The reagent member can also include surface active agents, such as for example, Tween 20 preferably at about 1%, or solvents, if necessary, to render it more hydrophilic, to promote rapid uptake of the liquid sample, and/or to facilitate the travel of the sample and the reagents though the porous carrier, and/or to minimize or to eliminate any interaction of the reagents or complexes with the porous carrier. Surface active agents can be incorporated in the solution containing the labeled reagent when this solution is applied to the reagent member during manufacture of the device or the component.

If the assay device is intended to identify more than one analyte in a single sample, the reagent member can incorporate several labeled specific binding reagents each carrying a different label, e.g., having different colors or fluorescent properties. This will facilitate the manufacture of a multiple analyte testing device.

The first color-labeled reagents are preferably incorporated in the reagent member in bulk, e.g., large sheet, form before being subdivided into individual members for use in a test device of the present invention. This is the case whether the reagent member is a separate component or a section of the porous carrier. Incorporation of the first color-labeled reagent in a separate reagent member avoids the need to apply first color-labeled labeled reagents in a special zone in the porous carrier, which may need careful pre-treatment. After a solution containing the first color-labeled reagent(s) has been allowed to saturate the reagent member, the reagent member may be dried, e.g., by vacuum or air-drying, or preferably by freeze-drying. Optionally, the solution can also contain a surface active agent, such as a detergent such as, for example, Tween 20 1% solution, and/or a glazing material, such as, for example, a sugar, e.g., sucrose. Their presence may enhance release of the first color-labeled reagents and may promote stability of certain specific binding reagents.

Incorporating the first color-labeled reagents in a separate reagent member(s), rather than pre-dosing onto the porous carrier that also incorporates the detection zone, enhances sensitivity of the test because a substantial quantity of the liquid sample is able to take up the first-color labeled reagent(s) before migrating through the porous carrier to the detection zone. Also, the liquid which permeates the carrier has a more uniform and consistent composition.

Preferably, the first and second color-labeled reagents are specific binding partners for the analyte. More preferably, the first and second color-labeled reagents bind specifically to different sites of the analyte. The first and second color-labeled reagents and the analyte (if present) cooperate together in a “sandwich” reaction. This results in the color-labeled reagents complexing in the detection zone if analyte is present in the sample. The two binding reagents should have specificities for different epitopes on the analyte.

The binding reagents are preferably highly specific antibodies, and more preferably, are monoclonal antibodies, including, but not limited to non-human, chimeric, and humanized monoclonal antibodies.

When the present invention is used as a test device for pregnancy, the two reagents specific for the analyte, hCG, preferably are monoclonal anti-hCG—clone “MIH 9816” from Seradyn and monoclonal anti-beta hCG—clone “057-10043” from OEM Concepts.

The label can be any entity the color and, therefore, the presence of which can be detected and, preferably, readily detected by the normal human eye. Concentration of the label or labels into a small zone or volume should give rise to a readily detectable signal such as, for example, a strongly-colored area. This can be evaluated by instruments if desired.

Preferably, the labels are a direct label, i.e., a label that can be used to produce an instant analytical result without the need to add further reagents in order to develop a detectable signal and/or, in its natural state, is readily visible either to the naked eye when it accumulates, or with the aid of an optical filter and/or applied stimulation, e.g. UV light to promote fluorescence. Preferably, the labels are particulate labels. Direct particulate labels including, but not limited to, colored latex particles and particularly those colors that are visually distinguishable from the natural color of the carrier or the material other than the second color-labeled specific binding reagents of the detection zone, gold sols, non-metallic colloids, and dye sols. They are robust and stable and can, therefore, be used readily in a analytical device which is stored in the dry state. Preferably, the particulate label is a latex particle, such as a colored latex particle. The first color and the second color can combine to form a third color that is detected. The particles and colors can be opaque, translucent, or a combination thereof to generate the third color if the first and second color-labeled particles complex with the analyte to be detected in the detection zone. If desired, the assay result can be read instrumentally, e.g., by color reflectance or microscopically. Alternatively, the labels can incorporate a fluorescent compound which can respond to applied electromagnetic energy such as ultraviolet light or visible light, to provide an emitted signal that can be measured instrumentally.

Indirect labels, such as enzymes, e.g. alkaline phosphatase and horseradish peroxidase, can be used.

Coupling of the color label to the specific binding reagent can be by covalent bonding, hydrophobic bonding, or other techniques known in the art. The first color-labeled reagent(s) migrate with the liquid sample as it progresses to the detection zone. Preferably, the flow of sample continues beyond the detection zone and sufficient sample is applied to test device in order that this may occur. Excess first color-labeled reagents which do not participate in forming any first complexes are flushed through the detection zone by this continuing flow.

Preferably, the porous carrier is nitrocellulose sheet having a pore size of at least about 1 micron, even more preferably of greater than about 5 microns, and yet more preferably about 8-12 microns. Nitrocellulose sheet having a nominal pore size of up to approximately 12 microns, is available commercially from Schleicher and Schuell GmbH.

Preferably, the nitrocellulose sheet is “backed”, e.g. with plastics sheet, to increase its handling strength. This can be manufactured easily by forming a thin layer of nitrocellulose on a sheet of backing material. The actual pore size of the nitrocellulose when backed in this manner will tend to be, lower than that of the corresponding unbacked material.

Alternatively, a pre-formed sheet of nitrocellulose can be tightly sandwiched between two supporting sheets of solid material, e.g. plastics sheets. I

It is preferable that the flow rate of an aqueous sample through the porous carrier is such that in the untreated material, aqueous liquid migrates at a rate of 1 cm in not more than 2 minutes, but slower flow rates can be used if desired.

The spatial separation between the reagent member and the detection zone, and the flow rate characteristics of the porous carrier, can be selected to allow adequate reaction times during which the necessary specific binding can occur. Further control over these parameters can be achieved by the incorporation of viscosity modifiers (e.g., sugars, modified celluloses, and surfactants) in the sample to slow down the reagent migration.

The second color-labeled specific binding reagent preferably is immobilized in the detection zone and, more preferably, is permanently immobilized there. The immobilized binding reagent can be impregnated throughout the thickness of the detection zone.

The presence or intensity of the signal from the first complexes which are retained in the detection zone can provide a qualitative or quantitative measurement of analyte in the sample. A plurality of detection zones arranged in series, through which the aqueous liquid sample can pass progressively, can also be used to provide a quantitative measurement of the analyte. Labels of different colors can be used to label specific binding reagents for different analytes to provide a multi-analyte test.

Preferably, the dry porous carrier material comprises a chromatographic strip, such as a strip of nitrocellulose. If desired, the nitrocellulose can be backed with moisture impermeable material, such as a polyester sheet. Using nitrocellulose as the porous carrier material has considerable advantage over more conventional strip materials, such as paper, because nitrocellulose has a natural ability to bind proteins without requiring prior sensitization. No chemical treatment is required which might interfere with the essential specific binding activity of the reagent. Nitrocellulose can be blocked using simple materials, such as polyvinylalcohol. Moreover, nitrocellulose is readily available in a range of pore sizes and this facilitates the selection of a porous carrier to suit particularly requirements such as sample flow rate.

The porous carriers can be manufactured individually, or may be in the form of a longer strip or a sheet during manufacture. The porous carrier can be manufactured in sheet form with the detection zone and the control zone applied to the sheet. The sheet can then be cut into strips.

The sample receiving member can be made from any bibulous, porous or fibrous material capable of absorbing liquid rapidly. The porosity of the material can be unidirectional (i.e., with pores or fibers running wholly or predominantly parallel to an axis of the member) or multidirectional (i.e., omnidirectional, so that the member has an amorphous sponge-like structure). Porous plastics material, such as polypropylene, polyethylene (preferably of very high molecular weight), polyvinylidene fluoride, ethylene vinylacetate, acrylonitrile and polytetrafluoro-ethylene can be used. It can be advantageous to pre-treat the member with a surface active agent during manufacture, as this can reduce any inherent hydrophobicity in the member and, therefore, can enhance its ability to take up and deliver a moist sample rapidly and efficiently. Sample receiving members can also be made from paper or other cellulosic materials, such as nitro-cellulose. Materials that are now used in the nibs of fiber-tipped pens are suitable and such materials can be shaped or extruded in a variety of lengths and cross-sections appropriate in the context of the invention. Preferably, the material comprising the sample receiving member should be chosen such that it and the reagent member can be saturated with aqueous liquid within a matter of seconds. Preferably, the material remains robust when moist, and for this reason paper and similar materials are less preferred in any embodiment wherein the sample receiving member protrudes from a housing. The liquid must thereafter permeate freely from the sample receiving member into the reagent member.

The control zone can be designed to convey an unrelated signal to the user that the device has worked. Various porous materials may be used for the porous barrier of the control zone. For example, different grades or concentrations of agarose can be used for different pore sizes. Alternatively, pores can be made in suitable materials by laser, drilling, microdrilling, or photoablation, for example. Porous barriers can be applied to the porous carrier material in a variety of ways including, but not limited to, printing techniques such as, for example, micro-syringes, pens using metered pumps, direct printing and ink-jet printing. Several different control zones can have different pore sizes to retain different sized first color-labeled specific binding reagents at each different porous barrier. Alternatively, the control zone can contain an anhydrous reagent that, when moistened, produces a color change or color formation, e.g. anhydrous copper sulphate which will turn blue when moistened by an aqueous sample. As a further alternative, a control zone could contain immobilized binding reagent or analyte which will react with excess labeled reagent(s). The control zone indicates to the user that the sample has proceeded past the detection zone and should be located downstream from the detection zone in which the desired test result is recorded. A positive control indicator, therefore, tells the user that the sample has permeated the required distance through the test device.

If desired, an absorbent sink or reservoir can be provided at the distal end of the test device. The absorbent sink or reservoir may comprise, for example, Whatman 3MM chromatography paper, and should provide sufficient absorptive capacity. Alternatively, a length of porous test device which may extend beyond the detection zone may act as a sink or reservoir.

The reagent member, porous carrier, and detection zone may be contained within a casing or housing, which is preferably composed of a moisture impervious material. The ample receiving member may extend out of the housing through an aperture and can act as a means for permitting a liquid sample to enter the housing and reach the reagent member. The housing should be provided with means, e.g., an appropriately placed aperture or window which may be open or covered, preferably with a transparent or translucent material, which enables the detection zone to be observable from outside the housing so that the result of the assay can be observed. The housing may also be provided with further means as above to enable the control zone to be observed from outside the housing. Preferably, the housing is provided with a removable cap or shroud which can protect the protruding porous receiving member during storage before use. If desired, the cap or shroud can be replaced over the protruding sample receiving member, after sample application, while the assay procedure is being performed. The casing can be constructed of opaque or translucent material incorporating at least one aperture through which the analytical result may be observed, together with a removable and replaceable cover for the protruding bibulous urine receiving member.

The present invention also provides an analytical method for detecting the presence or absence of an analyte. A device as set forth above is contacted with a liquid sample suspected of containing the analyte, such that the sample permeates by capillary action via the reagent member through the porous carrier into the detection zone and the first color-labeled reagent migrates therewith to the detection zone, the presence of analyte in the sample being determined by observing color change in the detection zone. The present invention can be used as a pregnancy testing device wherein the analyte is hCG and the color-labeled specific binding reagents are anti-hCG antibodies. A fertile period prediction device, essentially as just defined except that the analyte is LH and the labeled specific binding reagents are labeled anti-LH antibodies.

The devices of the present invention can be provided as kits suitable for home use, comprising a plurality (e.g., two or more) devices individually wrapped in moisture impervious wrapping and packaged together with appropriate instructions to the user.

If desired, a compound device according to the present invention can incorporate two or more test devices arranged in parallel and possibly sharing some components, for example, such that a single application of liquid sample to the device initiates sample flow in them simultaneously. Separate analytical results can be determined in this way. If different reagents are used on the test devices, the simultaneous determination of a plurality of analytes in a single sample can be made. Alternatively, multiple samples can be applied individually to an array of test devices and analyzed simultaneously.

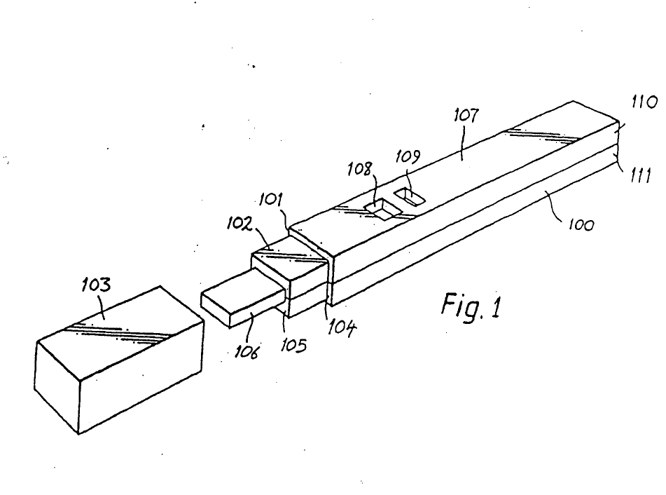

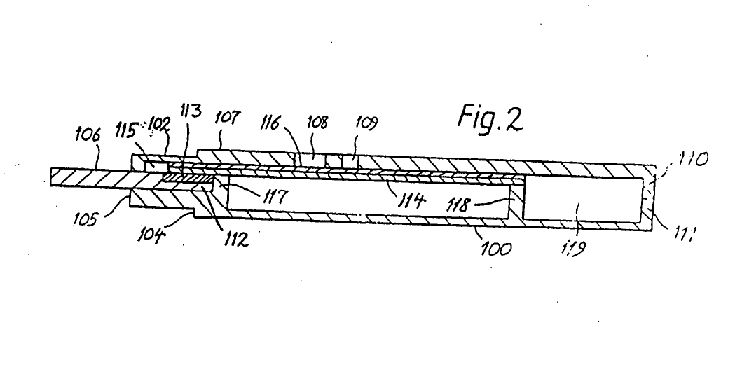

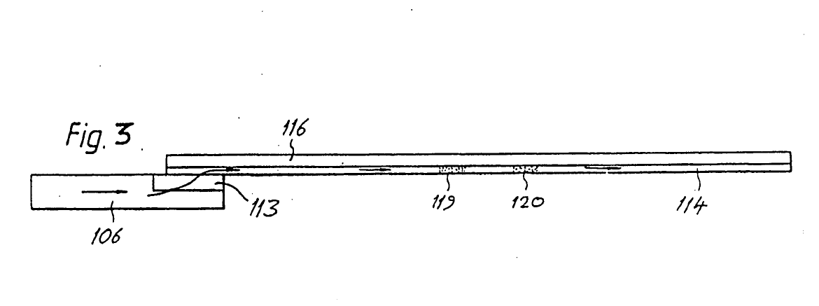

Referring to FIGS. 1-3, the device comprises a housing or casing 100 of elongate rectangular form having at one end 101 a portion 102 of reduced cross-sectional area. A cap 103 can be fitted onto portion 102 and can abut against the shoulder 104 at end 101 of the housing. Cap 103 is shown separated from housing 100. Extending beyond end 105 of portion 102 is a porous sample collector 106. When cap 103 is fitted onto portion 102 of the housing, it covers porous sample collector 106. Upper face 107 of housing 100 incorporates two apertures 108 and 109. The housing is constructed of an upper half 110 and a lower half 111.

Housing 100 is of hollow construction. Sample receiving member 106 extends into housing 100. The inner end 112 of sample receiving member 106 is recessed to accommodate a reagent member 113. Aqueous liquid sample applied to sample receiving member 106 can pass freely into reagent member 113, rapidly saturating it. In turn, reagent member 113 is in liquid permeable contact with porous carrier 114. The housing is constructed of an upper half 110 and a lower half 111 and porous carrier 114 overlaps reagent member 113 to ensure that there is adequate contact between these two components and that a liquid sample applied to sample receiving member 106 can permeate via reagent member 113 and into porous carrier 114. Porous carrier 114 extends further into housing 100. To help ensure that no liquid sample reaches porous carrier 114 without first passing through reagent member 113, a gap 115 can be left in the housing 100 by arranging for porous carrier 114 to overlap reagent member 113 only partially. Porous carrier 114 is backed by a supporting strip 116 formed of transparent moisture-impermeable plastics material. Porous carrier 114 extends beyond apertures 108 and 109. Means 117 and 118 are provided within housing 100 to hold porous carrier 114 firmly in place. The transparent backing strip 116 can act as a seal against undesired ingress of moisture from outside the housing 100. If desired, the residual space 119 within the housing can contain moisture-absorbant material, such as silica gel, to help maintain the strip 114 in the dry state during storage. The detection zone will lie in the region exposed through aperture 108 in order that when the device has been used, the result can be observed through aperture 108. Aperture 109 provides means through which a control zone containing may be observed.

In operation, the protective cap 103 is removed from the holder and sample receiving member 106 is exposed to a liquid sample e.g. by being placed in a urine stream in the case of a pregnancy test. After exposing sample receiving member 106 to the liquid sample for a time sufficient to ensure that the sample receiving member 106 is saturated with the sample, the cap 103 can be replaced and the device placed aside by the user for an appropriate period time (e.g. two or three minutes) while the sample permeates porous carrier 114 and the detection zone to provide the analytical result. After the appropriate time, the user can observe the result through apertures 108 and 109 and can ascertain whether the assay has been completed by observing the control zone 120 through aperture 109, and can ascertain the result of the assay by observing the detection zone 119 through aperture 108.

By using the components and reagents herein described, a device can be produced which is suitable for use as a pregnancy test kit or fertile period test kit for use in the home or clinic. The user merely needs to apply a urine sample to the exposed sample receiving member and then (after optionally replacing the cap) can observe the test result through aperture 108 within a matter of a few minutes. Although described with particular reference to pregnancy tests and fertile period tests, it will be appreciated that the device, as just described, can be used to determine the presence of a very wide variety of analytes if appropriate reagents are used. It will be further appreciated that aperture 109 is redundant and may be omitted if the test strip does not contain any control means. Further, the general shape of the housing and cap, both in terms of their length, cross-section and other physical features, can be the subject of considerable variation.

### EXAMPLE 1

Construction of a Test Device

A test device is constructed as follows:

Monoclonal anti-hCG—clone “MIH 9816” from Seradyn is conjugated on to a translucent bright red latex bead and monoclonal anti-beta hCG—clone “057-10043” from OEM Concepts is conjugated on to an opaque yellow latex bead, the beads having diameters of about 250 nm. A solution of the red-labeled antibody and Tween 20 1% solution is applied to a reagent member. A detection zone is prepared by immobilizing the yellow-labeled antibody in a downstream section of a nitrocellulose carrier strip. A control zone of an agarose matrix is applied to the carrier strip downstream of the detection zone. The pore size of the agarose matrix in the control zone is about 175 nm. A sample receiving member and a sink of absorbent material are provided. These components are assembled as in FIGS. 1-3 so that urine applied to the test device will flow sequentially from the sample receiving member to the reagent member to the porous carrier, through the detection zone and through the control zone to the sink.

### EXAMPLE 2

Detection of the Presence of hCG in Urine

The sample receiving member is saturated with urine containing hCG. The urine passes to the reagent member and solubilizes the red latex bead labeled antibodies and the surfactant. hCG complexes with some of the red latex bead labeled antibodies and this mixture travels downstream to the detection zone where the red latex bead labeled antibodies coupled with hCG complex with the immobilized yellow latex bead labeled antibodies, forming an orange line formed by the interaction of the red and yellow latex beads now immobilized in the three component complex in the detection zone. Excess uncomplexed red latex bead labeled antibodies travel in the urine to the control zone. The uncomplexed red latex bead labeled antibodies pass to the control zone. The uncomplexed red latex bead labeled antibodies are retained at the control zone and a red line appears. This test indicates the presence of hCG in the urine sample.

### EXAMPLE 3

Detection of the Absence of hCG in Urine

The sample receiving member is saturated with urine without hCG. The urine passes to the reagent member and solubilizes the red latex bead labeled antibodies and the surfactant. The uncomplexed red latex bead labeled antibodies travel in the urine to the porous carrier and to the detection zone. The uncomplexed red latex bead labeled antibodies pass through and to the control zone. The yellow color of the detection zone does not change. The uncomplexed red latex bead labeled antibodies are retained at the control zone and a red line appears. This test indicates the absence of hCG in the urine sample.

All patents, applications, literature references, and test methods cited herein are hereby incorporated by reference. All obvious modifications of the invention described herein are intended to fall within the scope of the invention as claimed.

1. A test device for detecting the presence or absence of a selected analyte in a liquid sample, said test device comprising:
a reagent member comprising a body and a first color-labeled binding reagent specific for a first binding site of said analyte; wherein said body is adapted to retain said first color-labeled specific binding reagent when said body and said first color-labeled binding reagent are dry, and to release said first color-labeled specific binding reagent when said body and said first color-labeled specific binding reagents are moist,;
a porous carrier; and
a detection zone comprising a second color-labeled binding reagent specific for a second binding site of said analyte, wherein said second color-labeled specific binding reagent is immobilized in said detection zone, wherein said first specific binding site and said second binding site are different, and wherein in said first and second labeled specific binding reagents are capable of forming a third color-labeled complex with said analyte, said complex comprising said first color-labeled specific binding reagent, said analyte, and said second color-labeled specific binding reagent;
wherein said first color, said second color, and said third color are visually distinguishable from one another by the human eye;
wherein said reagent member, porous carrier, and detection zone are arranged so that a fluid applied to said test device would travel sequentially from said reagent member to said porous carrier and to said detection zone.

2. A test device as defined in claim 1, wherein said test device is dry.

3. A test device as defined in claim 1 wherein said test device is moist.

4. A test device as defined in claim 1, wherein said detection zone is a section of said porous carrier.

5. A test device as defined in claim 1, wherein said detection zone is separate from and in fluid communication with said porous carrier.

6. A test device as defined in claim 1, further comprising a sample receiving member arranged so that a fluid applied to said test device would travel sequentially from said sample receiving member to said reagent member.

7. A test device as defined in claim 6, wherein said sample receiving member comprises a wick.

8. A test device as defined in claim 1, further comprising a control zone arranged so that a fluid applied to said test device would travel sequentially from said reagent member to said porous carrier, to said detection zone, and to said control zone.

9. A test device as defined in claim 8, wherein said control zone comprises a porous barrier having an average pore size smaller than the diameter of the first color-label.

10. A test device as defined in claim 8, wherein said control zone comprises a material that changes color when wet.

11. A test device as defined in claim 8, wherein said control zone comprises an immobilized binding reagent capable of binding to said first color-labeled specific binding reagent.

12. A test device as defined in claim 8, wherein said control zone is a section of said porous carrier.

13. A test device as defined in claim 8, wherein said control zone is separate from and in fluid communication with said porous carrier.

14. A test device as defined in claim 1, wherein said sample comprises a biological sample.

15. A test device as defined in claim 14, wherein said biological sample comprises urine.

16. A test device as defined in claim 14, wherein said biological sample comprises blood.

17. A test device as defined in claim 1, wherein said analyte comprises a protein.

18. A test device as defined in claim 17, wherein said protein comprises a hormone.

19. A test device as defined in claim 18, wherein said analyte comprises human chorionic gonadotrophin

20. A test device as defined in claim 18, wherein said analyte comprises human lutinizing hormone.

21. A test device as defined in claim 1, wherein said reagent body comprises a fibrous material.

22. A test device as defined in claim 22, wherein said regent member comprises a fiberglass pad.

23. A test device as defined in claim 1, wherein said reagent member comprises a porous material.

24. A test device as defied in claim 23, wherein said reagent member comprises a macroporous body.

25. A test device as defined in claim 1, wherein said label of said first color-labeled specific binding reagent comprises a direct label.

26. A test device as defined in claim 1, wherein said label of said first color-labeled specific binding reagent comprises an indirect label.

27. A test device as defined in claim 1, wherein said label of said first color-labeled specific binding reagent comprises a colored particle.

28. A test device as defined in claim 27, wherein said label of said first color-labeled specific binding reagent comprises a gold particle.

29. A test device as defined in claim 27, wherein said label of said first color-labeled specific binding reagent comprises a colored latex particle.

30. A test device as defined in claim 29, wherein said latex label is selected from the group consisting of a translucent colored latex particle and an opaque colored latex particle.

31. A test device as defined in claim 1, wherein said label of said second color-labeled specific binding reagent comprises a direct label.

32. A test device as defined in claim 1, wherein said label of said second color-labeled specific binding reagent comprises an indirect label.

33. A test device as defined in claim 1, wherein said label of said second color-labeled specific binding reagent comprises a colored particle.

34. A test device as defined in claim 33, wherein said label of said second color-labeled specific binding reagent comprises a gold particle.

35. A test device as defined in claim 33, wherein said label of said second color-labeled specific binding reagent comprises a colored latex particle.

36. A test device as defined in claim 35, wherein said label of said second color-label is selected from the group consisting of a translucent colored latex particle and an opaque colored latex particle.

37. A test device as defined in claim 1, wherein said first color-label is opaque and said second color-label is opaque.

38. A test device as defined in claim 1, wherein said first color-label is translucent and said second color-label is translucent.

39. A test device as defined in claim 1, wherein said first color-label is translucent and said second color-label is opaque.

40. A test device as defined in claim 1, said first color-label is opaque and said second color-label is translucent.

41. A test device as defined in claim 1, wherein said first specific binding reagent comprises a protein.

42. A test device as defined in claim 41, wherein said first specific binding reagent comprises an antibody.

43. A test device as defied in claim 42, wherein said first specific binding reagent comprises a monoclonal antibody.

44. A test device as defied in claim 43, wherein said first specific binding reagent comprises a non-human monoclonal antibody.

45. A test device as defied in claim 43, wherein said first specific binding reagent comprises a chimeric monoclonal antibody.

46. A test device as defined in claim 43, wherein said first specific binding reagent comprises a humanized monoclonal antibody.

47. A test device as defined in claim 43, wherein said antibody comprises an anti-hCG antibody.

48. A test device as defined in claim 1, wherein said second specific binding reagent comprises a protein.

49. A test device as defined in claim 1, wherein said second specific binding reagent comprises an antibody.

50. A test device as defied in claim 49, wherein said first specific binding reagent comprises a monoclonal antibody.

51. A test device as defied in claim 50, wherein said first specific binding reagent comprises a non-human monoclonal antibody.

52. A test device as defied in claim 50, wherein said first specific binding reagent comprises a chimeric monoclonal antibody.

53. A test device as defined in claim 50, wherein said first specific binding reagent comprises a humanized monoclonal antibody.

54. A test device as defined in claim 50, wherein said antibody comprises an anti-hCG antibody.

55. A test device as defined in claim 1, wherein said porous carrier comprises nitrocellulose.

56. A test device as defined in claim 1, further comprising a casing which contains at least a portion of said test device.

57. A test device as defined in claim 56, wherein said casing has an aperture from which said sample receiving member protrudes.

58. A test device as defined in claim 56, wherein said casing has a transparent or translucent window over at least a portion of said detection zone.

59. A test device as defined in claim 8, further comprising a casing which contains at least a portion of said test device.

60. A test device as defined in claim 59, wherein said casing has an aperture from which a sample receiving member protrudes, wherein said sample receiving member is arranged so that a fluid applied to said test device would travel sequentially from said sample receiving member to said reagent member.

61. A test device as defined in claim 60, wherein said casing has a transparent or translucent window over at least a portion of said detection zone.

62. A test device as defined in claim 61, wherein said casing has a transparent or translucent window over at least a portion of said control zone.

63. A test device as defined in claim 56, wherein said casing further comprises a removable cap adapted to cover said protruding sample receiving member.

64. A test device as defined in claim 59, wherein said casing further comprises a removable cap adapted to cover said protruding sample receiving member.

65. A test device for detecting the presence or absence of human chorionic gonadotrophin (hCG) in urine, said test device comprising:
a reagent member comprising a body, a first colored latex particle labeled anti-hCG antibody for a first antibody binding site of said hCG, wherein said reagent body is adapted to retain said first colored latex particle labeled anti-hCG antibody when said body and said first colored latex particle labeled anti-hCG antibody are dry, and to release said first colored latex particle labeled anti-hCG antibody when said body and said first colored latex particle labeled anti-hCG antibody are moist, and wherein in said first and second colored latex particle labeled anti-hCG antibodies are capable of forming a first labeled complex with said hCG, said first complex comprising said first colored latex particle labeled anti-hCG antibody, said hCG, and said second colored latex particle labeled anti-hCG antibody;
a nitrocellulose porous carrier; and
a detection zone comprising a second colored latex particle labeled anti-hCG antibody specific for a second antibody binding site of said hCG, wherein said first colored latex particle labeled anti-hCG antibody and said second colored latex particle labeled anti-hCG antibody binding site are different, wherein said second colored latex particle labeled anti-hCG antibody is immobilized in said detection zone, wherein said first antibody binding site and said second antibody site are different, and wherein said first and second colored latex particle labeled anti-hCG antibodies are capable of forming a third color-labeled complex with said hCG, said complex comprising said first colored latex particle labeled anti-hCG antibody, said analyte, and said second colored latex particle labeled anti-hCG antibody;
wherein said first color, said second color, and said third color are visually distinguishable from one another by the human eye;
wherein said reagent member, porous carrier, and detection zone are arranged so that urine applied to said test device would travel sequentially from said reagent member to said porous carrier and to said detection zone.

66. A test device as defined in claim 65, wherein said test device is dry.

67. A test device as defined in claim 65, wherein said test device is moist.

68. A test device as defined in claim 65, wherein said detection zone is a section of said porous carrier.

69. A test device as defined in claim 65, further comprising a sample receiving member arranged so that a fluid applied to said test device would travel sequentially from said sample receiving member to said reagent member.

70. A test device as defined in claim 65, further comprising a control zone arranged so that a fluid applied to said test device would travel sequentially from said reagent member to said porous carrier, to said detection zone, and to said control zone.

71. A method of detecting the presence or absence of an analyte in a liquid sample, said method comprising:
(a) applying said liquid sample to the reagent member of a test device as defined in claim 1; and
(b) detecting the presence or absence of said third color at said detection zone, wherein the presence of said third color in said detection zone indicates the presence of said analyte in said sample.

72. A test device for detecting the presence or absence of a selected analyte in a liquid sample, said test device comprising:
a reagent member comprising a body, a first color-labeled binding reagent specific for a first binding site of said analyte; wherein said body is adapted to retain said first color-labeled specific binding reagent when said body and said first color-labeled binding reagent are dry, and to release said first color-labeled specific binding reagent when said body and said first color-labeled specific binding reagents are moist,;
a porous carrier; and
a detection zone comprising a second color-labeled binding reagent specific for a second binding site of said analyte, wherein said second color-labeled specific binding reagent is immobilized in said detection zone, wherein said first specific binding site and said second binding site are different, and wherein in said first and second labeled specific binding reagents are capable of forming a third color-labeled complex with said analyte, said complex comprising said first color-labeled specific binding reagent, said analyte, and said second color-labeled specific binding reagent;
wherein said first color and said second colors are visually distinguishable from one another by the human eye and wherein said third color is the same as said first color;
wherein said reagent member, porous carrier, and detection zone are arranged so that a fluid applied to said test device would travel sequentially from said reagent member to said porous carrier and to said detection zone.
